(19) United States
(12) Patent Application Publication
(10) Pub. No.: US 2022/0275048 A1
(43) Pub. Date: Sep. 1, 2022

(54) DIMER IMMUNOADHESIN, PHARMACEUTICAL COMPOSTION AND USE THEREOF

(71) Applicant: PHARCHOICE THERAPEUTICS INC, Shanghai (CN)

(72) Inventors: Shi HU, Shanghai (CN); Min DING, Shanghai (CN)

(73) Assignee: PHARCHOICE THERAPEUTICS INC, Shanghai (CN)

(21) Appl. No.: 17/696,920

(22) Filed: Mar. 17, 2022

Related U.S. Application Data
(63) Continuation of application No. PCT/CN2020/112416, filed on Aug. 31, 2020.

(30) Foreign Application Priority Data
Sep. 18, 2019 (CN) 201910880542.5

(51) Int. Cl. C07K 14/725; C07K 14/705; C07K 14/715; C07K 14/72; A61P 15/08

(52) U.S. Cl. CPC C07K 14/7051; C07K 14/70521; C07K 14/7155; C07K 14/7151; C07K 14/7153; C07K 14/7158; C07K 14/7156; C07K 14/70578; C07K 14/72; A61P 15/08; A61K 38/00

(57) ABSTRACT

A soluble dimeric immunoadhesin includes a dimerized first polypeptide chain and a dimerized second polypeptide chain. The first polypeptide chain has a general formula of Z1-Z2, and the second polypeptide chain has a general formula of Y1-Y2. Z1 is (i) an extracellular domain of a first cell surface receptor or a functional variant or fragment thereof, or (ii) a first cytokine or a functional variant or fragment thereof; Z2 is a dimerization domain of an immunoglobulin constant region or a functional variant or fragment thereof. Y1 is an extracellular domain of a second cell surface receptor or a functional variant or fragment thereof, or (ii) a second cytokine or a functional variant or fragment thereof. Y2 is a dimerization domain of an immunoglobulin constant region or a functional variant or fragment thereof. A dimeric protein can be used for the treatment and prevention of infertility-related diseases.

### CROSS-REFERENCE TO RELATED APPLICATIONS

This application is a continuation of International Patent Application No. PCT/CN2020/112416 with a filing date of Aug. 31, 2020, designating the United States, now pending, and further claims priority to Chinese Patent Application No. 201910880542.5 with a filing date of Sep. 18, 2019. The content of the aforementioned applications, including any intervening amendments thereto, are incorporated herein by reference.

### TECHNICAL FIELD

The present disclosure relates to the technical field of biomedicine engineering, in particular to a dimeric immunoadhesin, a pharmaceutical composition using the dimeric immunoadhesin as an active component and medical use thereof, especially use thereof in the treatment of infertility-related diseases.

### BACKGROUND ART

There are a plurality of important membrane molecules on the surface of T cells, which play an important role in the activation, proliferation and differentiation of T cells and the exertion of effector functions. According to the functions, they can be divided into the following categories: (1) TCR-CD3 complex, which enables T cells to recognize the antigen peptide-WIC molecular complex on the antigen-presenting cell and transmit activation signals to the cell; (2) CD4 and CD8 molecules, which assist the TCR of CD4+ and CD8+ T cells to recognize antigens and participate in the transduction of T cell activation signals, respectively; (3) costimulatory molecules: such as CD28, CTLA-4, ICOS and PD-1, which transmit the second signal to T cells; (4) other surface molecules, which mainly include cytokine receptors related to T cell activation, proliferation and differentiation, and adhesion molecules that interact with cells.

TIGIT protein (UniProtKB code: Q495A1) is a newly discovered costimulatory molecule with immunosuppressive effect in recent years. In 2005, Abbas et al. (Abbas A R, Baldwin D, Ma Y, et al. Genes & Immunity, 2005, 6(4): 319-331) sequenced activated human T cells in order to look for new costimulatory or inhibitory molecules, and further investigated some protein molecules with immunomodulatory domains. As a result, a new molecule expressed on T cells and NK cells was discovered. This molecule has an immunoglobulin-like domain, a transmembrane domain and an immunoreceptor tyrosine-based inhibitory motif (ITIM), which is therefore named T cell immunoglobulin and ITIM domain (TIGIT) (Xin Y, Harden K, Gonzalez L C, et al. Nature Immunology, 2009, 10(1):48-57). Soon, other laboratories also used different methods to find this molecule, and individually named WUCAM (Boles K S, Vermi W, Facchetti F, et al., 2010, 39(3): 695-703), Vstm3 (Levin S D, Taft D W, Brandt C S, et al. Vstm3 is a member of the CD28 family and an important modulator of T-cell function.[J]. European Journal of Immunology, 2011, 41(4):902-915) or Vsig9 (Stanietsky N, Mandelboim O. Paired NK cell receptors controlling NK cytotoxicity[J]. Febs Letters, 2010, 584(24):4895-4900).

In the prior art, soluble fragments of TIGIT protein have been confirmed in basic research to have a certain inhibitory effect on antigen presentation of antigen-presenting cells such as DC at a cellular level (Xin Y, Harden K, Gonzalez L C, et al. Nature Immunology, 2009, 10(1):48-57), and can be used in the treatment of autoimmune diseases such as lupus nephritis (Liu S, Sun L, Wang C, et al. Clinical immunology. 2019; 203: 72-80).

But in fact, a plurality of cell surface receptors are known to have a similar soluble form to the TIGIT protein. These soluble receptors correspond to the ligand binding domains of their cell surface counterparts. For example, naturally occurring soluble cytokine receptors inhibit cytokine responses and act as transport proteins. In addition, it has been found that soluble receptor polypeptides are dimerized by using fusion proteins to enhance the binding properties of these soluble receptors, so that they become therapeutically useful antagonists of their corresponding ligands. Representatives of such dimeric fusion bodies are immunoadhesins. (See, for example, Sledziewski et al, U.S. Pat. Nos. 5,155,027 and 5,567,584; Jacobs et al, U.S. Pat. No. 5,605,690; Wallner et al, U.S. Pat. No. 5,914,111; and Ashkenazi and Chamow, Curr. Opin. Immunol. 9:195-200, 1997).

However, in recurrent abortion, the imbalance of maternal-fetal immune factors is also a key link in the pathological progression of the disease (Trowsdale J, Betz A G. Nat Immunol. 2006; 7: 241-6). But due to the particularity of intrauterine maternal-fetal immunity, the therapeutic value of these immunoadhesins is still unclear. The present disclosure described herein clarifies the application value of such drugs.

### SUMMARY

An objective of the present disclosure is to rely on the above background art to study whether soluble dimer immunoadhesins can be used in the treatment of recurrent abortion, threatened abortion and other related diseases mediated by maternal-fetal immune disorders, and to describe the specific structure, preparation method and use of the dimeric immunoadhesions. That is, the present disclosure provides a dimeric immunoadhesin, a preparation method and use thereof.

A first aspect of the present disclosure provides a soluble dimeric immunoadhesin. The soluble dimeric immunoadhesin includes a dimerized first polypeptide chain and a dimerized second polypeptide chain, the first polypeptide chain has a general formula of Z1-Z2, and the second polypeptide chain has a general formula of Y1-Y2. Z1 is (i) an extracellular domain of a first cell surface receptor or a functional variant or fragment thereof, or (ii) a first cytokine or a functional variant or fragment thereof; Z2 is a dimerization domain or a functional variant or fragment thereof. Y1 is (i) an extracellular domain of a second cell surface receptor or a functional variant or fragment thereof, or (ii) a second cytokine or a functional variant or fragment thereof; Y2 is a dimerization domain or a functional variant or fragment thereof.

In certain embodiments of the above polypeptide chain or dimeric immunoadhesin (where Z1 is an extracellular domain of a first cell surface receptor or a functional variant or fragment thereof, and/or Y1 is an extracellular domain of a second cell surface receptor or a functional variant or fragment thereof), the first cell surface receptor and/or the second cell surface receptor may be each being any one selected from the group consisting of: 4-1BB; ACTH receptor; activin receptor; BLTR (leukotriene B4 receptor); BMP receptor; C3a receptor; C5a receptor; CCR1; CCR2; CCR3; CCR4; CCR5; CCR6; CCR7; CCR8; CCR9; CD19; CD22; CD27; CD28; CD30; CD40; CD70; CD80; CD86; CD96; CD200R; CTLA-4; CD226; CD274; CD273; CD275; CD276; CD278; CD279; VSTM3 (TIGIT, B7R1); CD112; CD155; B7H6; NKp30; ICAM; VLA-4; VCAM; CT-1 receptor; CX3CR1; CXCR1; CXCR2; CXCR3; CXCR4; CXCR5; D6; DARC; DcR3; DR4; DR5; DcR1; DcR2; ECRF3; Fas; fMLP receptor; G-CSF receptor; GIT receptor; GM-CSF receptor; growth hormone receptor; HVEM; BTLA; interferon-α receptor; interferon-β receptor; interferon-γ receptor; IL-1 receptor type I; IL-1 receptor type II; IL-10 receptor; IL-11 receptor; IL-12 receptor; IL-13 receptor; IL-15 receptor; IL-16 receptor (CD4); IL-17 receptor A; IL-17 receptor B; IL-17 receptor C; IL-17 receptor D; IL-17 receptor E; IL-18 receptor; IL-2 receptor; IL-3 receptor; IL-4 receptor; IL-5 receptor; IL-6 receptor; IL-7 receptor; IL-9 receptor; IL-20 receptor A; IL-20 receptor B; IL-21 receptor; IL-22 receptor A; IL-22 receptor B; IL-28 receptor A; IL-27 receptor A; IL-31 receptor A; BCMA; TACI; BAFF receptor; immunomodulatory semaphoring receptor CD72; Kaposi's sarcoma-associated herpesvirus GPCR; lipoxin A4 receptor; lymphotoxin β receptor; lysophospholipid growth factor receptor; neurokinin 1; μ-, δ-, and κ-opioid receptors of endorphins; oncostatin M receptor; osteopontin receptor; osteoprotegerin; Ox40; OX40L; PACAP and VIP receptors; PAF receptor; poxvirus; IFNα/β receptor homologs; poxvirus IFNγ receptor homologs; poxvirus IL-10 receptor homologs; poxvirus membrane-bound G protein-coupled receptor homologs; poxvirus-secreted chemokine binding protein; poxvirus TNF receptor homologs; prolactin receptor; RANK; RON receptor; SCF receptor; somatostatin receptor; T1/ST2; TGF-β receptor; TNF receptor (for example, p60 and p80); TNFRSF19; TPO receptor; US28; XCR1; erythropoietin receptor; growth hormone receptor; leukemia inhibitory factor receptor; and C-kit receptor.

In the case where both the Z1 and the Y1 are extracellular domains of cell surface receptors or functional variants or fragments thereof, the first and the second cell surface receptors may be the same or different.

In other embodiments, in the case where the Z1 is a first cytokine or a functional variant or fragment and/or the Y1 is a second cytokine or a functional variant or fragment thereof, the first cytokine and/or the second cytokine may be each selected from the group consisting of: α-MSH; 9E3/cCAF; ACTH; activin; AK155; angiogenesis inhibitor; Apo2L/TRAIL; APRIL; BAFF (BLys); BLR1 ligand/BCA-1/BLC/CXCL13; BMP family; BRAK; calcitonin gene-related peptide (CGRP); molluscum contagiosum virus CC chemokine; CCL27; CCL28; CD100/Sema4D; CD27 ligand; CD30 ligand; CD40 ligand; CK08-1/MPIF-1/CCL23; CLF/CLC; CSF-1; CT-1; CTAP-III, βTG and NAP-2//CXCL7; CXCL16; defensins; ELC/MIP-30/Exodus-3/CCL19; ENA-78/CXCL5; endorphins; endostatin; eosinophil chemotactic factor 2/MPIF-2/CCL24; eosinophil chemotactic factor/CCL11; erythropoietin; Exodus-1/LARC/MIP-3a (SCYA20); Fas ligand; Flt-3 ligand; fMLP; Fractalkine/CX3CL1; G-CSF; GCP-2/CXCL6; GM-CSF; growth hormone; HCC-1/CCL14; HCC-4/CCL16; high-mobility group box 1 (HMGB1); human cathelicidin antimicrobial peptide LL-37; I-309/CCL1; IFNα, IFNβ and IFNω ligands; IFNγ; IL-1α; IL-1β; IL-10; IL-11; IL-12; IL-13; IL-15; IL-16; IL-17A; IL-17B; IL-17C; IL-17D; IL-17E; IL-17F; IL-18; IL-1Ra; IL-2; IL-27; IL-3; IL-4; IL-5; IL-6; IL-7; IL-8/CXCL8; IL-9; IP-10/CXCL10; IL-19; IL-20; IL-21; IL-22; IL-23; IL-24; IL-26; IL-31; keratinocyte growth factor; KSHV-associated IL-6 ligand; leptin; leukotaxin 1/HCC-2/MIP-1δ/CCL15; leukotriene B4; LIGHT; lipoxin; chemotactic factor for lymphocyte (ChFL)/XCL1; lymphotoxins a and (3; lysophospholipid growth factor; macrophage-derived chemokine; macrophase-stimulating protein (MSP); MCP-1/CCL2, MCP-2/CCL8, MCP-3/CCL7, MCP-4/CCL13, and MCP-5/CCL12; methoxyestradiol; MGSA/GRO/CXCL1, CXCL2, and CXCL3; MIF; MIG/CXCL9; MIP-1a/CCL3 and MIP-1β/CCL4; MIP-1γ/MRP-2/CCF18/CCL9/10; MuC10/CCL6; oncostatin M; osteopontin; parapoxvirus (orf virus) IL-10 homologs; PARC/DC-CCK1/AMAC-1/CCL18; PDGF-A; PDGF-B; PDGF-C; PDGF-D; platelet activating factor; platelet factor 4/CXCL4; poxvirus growth factor related to epidermal growth factor; poxvirus-secreted complement regulatory protein; poxvirus vascular endothelial growth factor (VEGF) homologs of orf virus; prolactin; RANK ligand; RANTES/CCL5; S100A12; SDF-1/CXCL12; SERP-1, secreted poxvirus serine protease inhibitor; SLC(6Ckine)/Exodus-2/TCA-4/CCL21; somatostatin; stem cell factor; substance P; TARC/CCL17; TCA3/mouse CCL1; TECK/CCL25; TGFβ; thrombopoietin; TNFα; TSG-6; TWEAK; vaccinia virus semaphorin; vCXC-1 and vCXC-2; VEGF; VIP and PACAP; and viral IL-10 variants.

In the case where both the Z1 and the Y1 are cytokines or functional variants or fragments thereof, the first and the second cytokines may be the same or different.

Particularly suitable dimerization domains used in accordance with the foregoing dimeric immunoadhesins may include immunoglobulin (IgG) heavy chain constant regions. For example, in a specific variation, the dimerization domains Z2 and Y2 are Fc fragments of IgG, such as human immunoglobulin yl Fc fragment. When the Z1 is different from the Y1, the dimerization domains Z2 and Y2 may be engineered to increase the formation of specific heterodimerization, such as Knob-in-holes, ART-Ig that changes charge polarity, BiMab, and other bispecific antibody constant region construction and engineering methods (review literature: Brinkmann U, Kontermann R E. mAbs, 2017, 9(2): 182-212).

In some embodiments of the foregoing dimeric immunoadhesin, the dimerization domains Z2 and Y2 include a peptide linker, and the peptide linker consists of 15-32 amino acid residues, 1 to 8 (for example, 2) of are cysteine residues. In a specific variation, the Z2 and the Y2 contain an immunoglobulin hinge region or a variant thereof. For example, in a specific example, the Z2 and the Y2 contain an immunoglobulin hinge variant (for example, a human immunoglobulin yl hinge variant), in which a cysteine residue at position 220 of the Fc fragment is replaced by serine. Particularly suitable peptide linkers used in accordance with the foregoing dimerization domains Z2 and Y2 include such peptide linkers that include a plurality of glycine residues, and optionally at least one serine residue.

In certain embodiments of the present disclosure, the dimerization domains Z2 and Y2 may be active variants of the Fc fragment of human immunoglobulin, such as using an Fc domain of IgG2, IgG3, or IgG4. In some embodiments, Fc mutants may be further used to reduce biological activities of immunoglobulins such as ADCC and complement fixation, for example, LALA-PG mutant, L235E; E318A; K320A; K322A mutant, and the like.

In a preferred embodiment of the present disclosure, each of the Z1 and the Y1 is an extracellular domain of TIGIT (VSTM3, B7R1) or a functional variant or fragment thereof. For example, in specific variations of the soluble dimeric immunoadhesins having the foregoing general formulas Z1-Z2 and Y1-Y2, the amino acid sequences of the Z1 and the Y1 are at least 60%, preferably at least 65%, preferably at least 70%, more preferably at least 75%, yet more preferably at least 80%, still more preferably at least 85%, even more preferably at least 90%, yet even more preferably at least 95%, and most preferably at least 99% identical to an amino acid sequence of a human TIGIT protein shown in SEQ ID NO: 1 at positions 22-141.

In a specific preferred embodiment, in a specific variation of a dimeric immunoadhesin containing the general formulas Z1-Z2 and Y1-Y2 (and where each of the Z1 and the Z2 is an extracellular domain of TIGIT or a functional variant or fragment thereof), the dimeric immunoadhesin may include the following selected amino acid sequence: an amino acid sequence of human TIGIT immunoadhesin shown in SEQ ID NO: 2.

In a specific preferred embodiment, in a specific variation of a dimeric immunoadhesin containing the general formulas Z1-Z2 and Y1-Y2 (and where each of the Z1 and the Z2 is an extracellular domain of TIGIT or a functional variant or fragment thereof), the dimeric immunoadhesin may include the following selected amino acid sequence: an amino acid sequence of an LALA-PG variant of human TIGIT immunoadhesin shown in SEQ ID NO: 3.

In a preferred embodiment of the present disclosure, the Z1 is an extracellular domain of TIGIT or a functional variant or fragment thereof. The Y1 is an extracellular domain of CTLA4 or a functional variant or fragment thereof. For example, in specific variations of the soluble dimeric immunoadhesins having the foregoing general formulas Z1-Z2 and Y1-Y2, the amino acid sequence of the Z1 is at least 60%, preferably at least 65%, preferably at least 70%, more preferably at least 75%, yet more preferably at least 80%, still more preferably at least 85%, even more preferably at least 90%, yet even more preferably at least 95%, and most preferably at least 99% identical to the amino acid sequence shown in SEQ ID NO: 1. The amino acid sequence of the Y1 is at least 60%, preferably at least 65%, preferably at least 70%, more preferably at least 75%, yet more preferably at least 80%, still more preferably at least 85%, even more preferably at least 90%, yet even more preferably at least 95%, and most preferably at least 99% identical to an amino acid sequence of an N-terminal domain of ABATACEPT shown in SEQ ID NO: 4.

In a specific preferred embodiment, in a specific variation of a soluble dimeric immunoadhesin containing the general formulas Z1-Z2 and Y1-Y2 (where the Z1 is an extracellular domain of TIGIT or a functional variant or fragment thereof; the Y1 is an extracellular domain of CTLA4 or a functional variant or fragment thereof), the two polypeptide chains of the soluble dimer immunoadhesin may include the following selected amino acid sequences: (a) the Z1-Z2 polypeptide chain includes an amino acid sequence shown in SEQ ID NO: 5, and (b) the Y1-Y2 polypeptide chain includes an amino acid sequence shown in SEQ ID NO: 6.

In a preferred embodiment of the present disclosure, the Z1 is an extracellular domain of TIGIT or a functional variant or fragment thereof. The Y1 is cytokine IL-10 or a functional variant or fragment thereof. For example, in specific variations of the soluble dimeric immunoadhesins having the foregoing general formulas Z1-Z2 and Y1-Y2, the amino acid sequence of the Z1 is at least 60%, preferably at least 65%, preferably at least 70%, more preferably at least 75%, yet more preferably at least 80%, still more preferably at least 85%, even more preferably at least 90%, yet even more preferably at least 95%, and most preferably at least 99% identical to the amino acid sequence shown in SEQ ID NO: 1. The amino acid sequence of the Y1 is at least 60%, preferably at least 65%, preferably at least 70%, more preferably at least 75%, yet more preferably at least 80%, still more preferably at least 85%, even more preferably at least 90%, yet even more preferably at least 95%, and most preferably at least 99% identical to an amino acid sequence shown in SEQ ID NO: 7.

In a specific preferred embodiment, in a specific variation of a soluble dimeric immunoadhesin containing the general formulas Z1-Z2 and Y1-Y2 (where the Z1 is an extracellular domain of TIGIT or a functional variant or fragment thereof; the Y1 is an extracellular domain of the cytokine IL-10 or a functional variant or fragment thereof), the two polypeptide chains of the soluble dimer immunoadhesin may include the following selected amino acid sequences: (a) the Z1 is the amino acid sequence shown in SEQ ID NO: 5, and (b) the Y1 is a mutated amino acid sequence shown in SEQ ID NO: 8.

In addition, the present disclosure further provides a polynucleotide, which encodes the foregoing dimeric immunoadhesin. In a related aspect, the present disclosure provides a vector containing such a polynucleotide. For example, in some embodiments, the present disclosure provides an expression vector including the following operably linked elements: a transcription promoter; a DNA region encoding the foregoing dimeric immunoadhesin; and a transcription terminator.

In other related aspects, the present disclosure provides cultured cells containing the vector, and methods for producing the polypeptides or dimeric proteins as disclosed above. For example, in some embodiments, the cultured cell according to the present disclosure includes an expression vector, and the expression vector contains the following operably linked elements: a transcription promoter; a DNA segment encoding the foregoing dimeric immunoadhesin; and a transcription terminator; and the cells express the dimeric immunoadhesin encoded by the DNA segment. In some variations of a preparation method of the dimeric immunoadhesin, the method includes the following steps: (i) culturing cells containing the expression vector as disclosed above, where the cells express the dimeric immunoadhesin encoded by the DNA segment and produce the encoded dimeric immunoadhesin; and (ii) recovering the soluble dimeric immunoadhesin. Similarly, in some variations of a preparation method of a dimeric protein, the method includes the following steps: (i) culturing cells containing the expression vector as disclosed above, where the cells express the dimeric immunoadhesin encoded by the DNA segment, and produce the encoded dimeric immunoadhesin as the dimeric protein; and (ii) recovering the dimeric protein.

A second aspect of the present disclosure provides a pharmaceutical composition. The pharmaceutical composition includes the foregoing soluble dimeric immunoadhesin and at least one pharmaceutically acceptable carrier. Thus, more stable efficacy may be exerted. These formulations may ensure the conformational integrity of a core amino acid sequence of a TIGIT immunoadhesin disclosed in the present disclosure, and protect multifunctional groups of a protein to prevent degradation (including but not limited to aggregation, deamination, or oxidation) thereof.

Normally, liquid formulations may be stored stably at 2-8° C. for at least one year, and lyophilized formulations may be stable at 30° C. for at least six months. The formulations may be suspensions, injections, and lyophilized preparations commonly used in the pharmaceutical field, and preferably injections or lyophilized preparations.

For the water injections or lyophilized preparations of the dimeric immunoadhesin disclosed in the present disclosure, pharmaceutically acceptable excipients include one or a combination of surfactants, solution stabilizers, isotonic regulators, and buffers. Herein, the surfactants include nonionic surfactants such as polyoxyethylene sorbitan fatty acid esters (Tween 20 or 80); poloxamers (such as poloxamer 188); Triton; sodium dodecyl sulfate (SDS); sodium lauryl sulfate (SLS); tetradecyl, linoleoyl or octadecyl sarcosine; Pluronics; and MONAQUAT™, and adding quantity thereof should minimize the tendency of a bifunctional bispecific antibody protein to granulate. The solution stabilizers may be saccharides including reducing sugars and non-reducing sugars, amino acids including monosodium glutamate or histidine, and alcohols including one or a combination of trihydric alcohols, higher sugar alcohols, propanediol, and polyethylene glycol; adding quantity of the solution stabilizers should enable a finally formed formulation to remain stable within a time considered by those skilled in the art to reach a stable state; the isotonic regulators may be one of sodium chloride and mannitol; the buffer may be one of TRIS, histidine buffer, and phosphate buffered saline (PBS).

A third aspect of the present disclosure provides use of the foregoing dimeric immunoadhesin, and provides use of the dimeric immunoadhesin in the preparation of a medicament for the treatment and prevention of infertility-related diseases. The medicament adopts the soluble immunoadhesin protein as disclosed above as an active component. Administration methods include: administering an effective dosage of the soluble immunoadhesin protein to subjects (human or animals) with the infertility-related diseases, or prophylactically administering an effective dosage of the soluble immunoadhesin protein to healthy subjects at risk of infertility.

In some preferred embodiments of the present disclosure, infertility-related diseases suitable for using the soluble immunoadhesin disclosed herein may include diseases related to maternal-fetal immune tolerance disorder and gynecological reproductive inflammation. The former may include recurrent spontaneous abortion, threatened abortion, or treatment failure of assisted reproductive technology; the latter may include pelvic inflammatory disease, decreased endometrial receptivity, endometritis, endometrial polyps, intrauterine adhesions, reduction of endometrial glands, endometrial fibrosis, amenorrhea, abnormal uterine bleeding, adenomyosis and endometriosis, reproductive system infection, and hysteromyoma.

Through the classic verification experiment of an abortion model of maternal-fetal immune tolerance disorder, the dimeric immunoadhesin may significantly reduce the abortion rate; through the endometrial injury model verification test, dimeric immunoadhesin therapy may effectively alleviate the endometrial injury caused by uterine aspiration, and effectively relieve the formation of endometrial and subendometrial fibrotic tissue and improve endometrial receptivity.

The Present Disclosure has the Following Beneficial Guarantees and Effects:

The dimeric immunoadhesin, pharmaceutical composition and use provided by the present disclosure have simple construction and expression processes. It is proved experimentally that the dimeric immunoadhesin has excellent therapeutic effects on diseases related to maternal-fetal immune tolerance disorder and gynecological reproductive inflammation. Administration of the dimeric immunoadhesin alone or in combination with other drugs for related diseases may effectively treat related diseases caused by maternal-fetal immune disorders, and has broad clinical application prospects.

### BRIEF DESCRIPTION OF THE DRAWINGS

FIG. 1 is a schematic diagram of the structure of TIGIT immunoadhesin;

FIG. 2 illustrates an effect of a plurality of soluble dimeric immunoadhesins on the secretion of IL-10 and TNFα in decidual dendritic cells;

FIG. 3 illustrates a therapeutic effect of administration of a plurality of soluble dimeric immunoadhesins in a mouse model of immune spontaneous abortion;

FIG. 4 illustrates an effect of soluble dimeric immunoadhesin on the expression of T helper cells in mouse para-aortic lymph nodes;

FIG. 5 illustrates an effect of soluble dimeric immunoadhesin on pregnant mouse endometrial receptivity markers LIF and OSM;

FIG. 6 illustrates an effect of soluble dimeric immunoadhesin on the degree of endometrial fibrosis in mice.

### DETAILED DESCRIPTION OF THE EMBODIMENTS

The following examples and experimental examples further illustrate the present disclosure, and should not be construed as limiting the present disclosure. The examples do not include detailed descriptions of conventional methods, such as those used for constructing vectors and plasmids, those for inserting protein-encoding genes into such vectors and plasmids, or those for introducing plasmids into host cells. Such methods are well known to those of ordinary skills in the art, and have been described in a plurality of publications, including Sambrook, J., Fritsch, E. F. and Maniais, T. (1989) Molecular Cloning: A Laboratory Manual, 2^{nd} edition, Cold spring Harbor Laboratory Press.

### Example 1. Construction and Expression of Soluble Dimeric Immunoadhesins

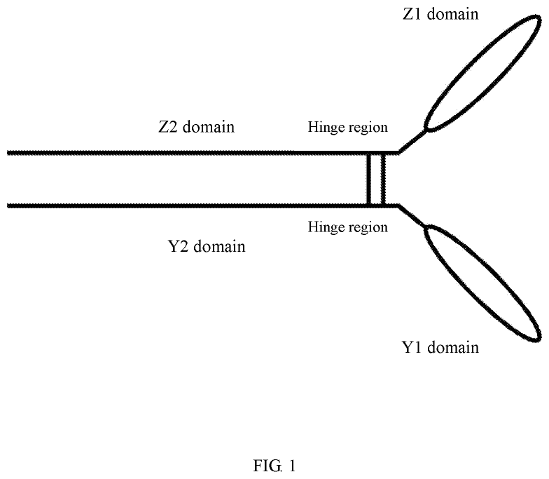

As shown in FIG. 1, soluble dimeric immunoadhesin is a dimer with antibody IgG Fc. The method for constructing and expressing dimeric immunoadhesin itself is a conventional experimental technique in the field. A briefly description is as follows:

(1) Full gene synthesis was used to synthesize soluble dimeric immunoadhesins: TIGIT-Fc-wt (containing two polypeptide chains; the amino acid sequence and nucleotide sequence of each polypeptide chain are shown in SEQ ID NO: 2 and SEQ ID NO: 9); TIGIT-Fc-LALA-PG (containing two polypeptide chains; the amino acid sequence and nucleotide sequence of each polypeptide chain are shown in SEQ ID NO: 3 and SEQ ID NO: 10); TIGIT/CTLA4-Fc (containing two polypeptide chains; the amino acid sequence and nucleotide sequence of the first polypeptide chain are shown in SEQ ID NO: 5 and SEQ ID NO: 11, and those of the second polypeptide chain are shown in SEQ ID NO: 6 and SEQ ID NO: 12); TIGIT/IL10-Fc (containing two polypeptide chains; the amino acid sequence and nucleotide sequence of the first polypeptide chain are shown in SEQ ID NO: 5 and SEQ ID NO: 11, and those of the second polypeptide chain are shown in SEQ ID NO: 8 and SEQ ID NO: 13).

(2) Expression and Purification of Fusion Proteins

The soluble dimeric immunoadhesins were expressed according to the method as described in the literature (Finck B K. Science, 265; Mihara M et al. Journal of Clinical Investigation. 2000; 106: 91-101; Yu X, et al. Nature Immunology. 2009; 10: 48-57. Liu S, et al. Clin Immunol. 2019 June; 203:72-80).

### Example 2. Biacore Analysis

Biacore T100 (GE Healthcare) was used to detect the affinity of each immunoadhesin according to the method in the literature (Bruhns P. et al. Blood, 2009, 113(16): 3716-3725). The specific values of the detected affinity are shown in Table 1.

TABLE 1
Biacore analysis results (in nM)

| | TIGIT- | TIGIT-Fc- | TIGIT/ | TIGIT/ |
|---|---|---|---|---|
| Parameter | Fc-wt | LALA-PG | CTLA4-Fc | IL10-Fc |
| Affinity/kinetics | 1.55 | 1.84 | 2.39 | 2.41 |
| of human CD155 | | | | |
| Affinity/kinetics | 2.07 | 2.15 | 3.55 | 3.21 |
| of mouse CD155 | | | | |
| Affinity/kinetics | — | — | 2.51 | — |
| of B7 | | | | |
| Affinity/kinetics | — | — | — | 0.91 |
| of anti-IL 10 | | | | |

### Example 3. The Effect of Dimeric Immunoadhesin on Decidual Immune Cells

Dendritic cells (DCs) (CD1c positive) were isolated from human decidual tissue that terminated pregnancy for non-medical reasons. Isolation and screening methods were the same as those in the literature (Guo P F, et al. Blood, 2010, 116(12): 2061-2069). The DC cells were divided into a negative control group (control IgG, 10 μg/mL), a dimeric immunoadhesin treatment group (dimeric immunoadhesin, 10 μg/mL), and LPS treatment group (100 ng/mL). The levels of interleukin 10 (IL-10) and tumor necrosis factor α (TNFα) were detected after 48 h. The detection method was the same as that in the literature (Guo P F, et al. Blood, 2010, 116(12): 2061-2069).

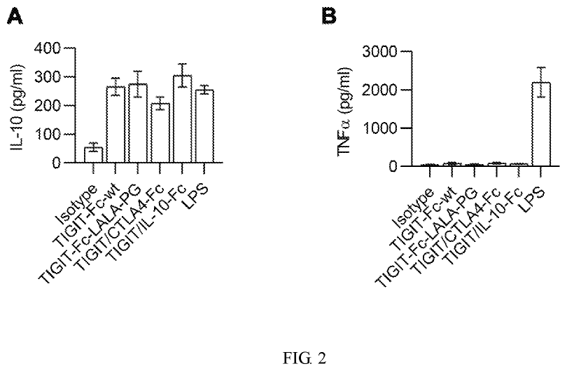

The detection results are shown in FIG. 2, showing that the dimeric immunoadhesin can significantly increase the secretion level of IL-10 without increasing the level of TNFα, and demonstrating that the dimeric immunoadhesin can exert immune tolerance through DCs.

### Example 4. Therapeutic Effect of Dimeric Immunoadhesin on Spontaneous Abortion Model

Female CBA/J mice and male DBA/2J mice were used to establish a stress abortion model. This abortion model was a classic research model of maternal-fetal immune tolerance disorder. Its establishment method, experimental method and observation time points were the same as those in the literature (Blois S M, et al. Nature Medicine, 2007, 13(12): 1450-1457).

The mice were divided into a negative control group, a stress group, and a dimeric immunoadhesin treatment group immediately after confirming that vaginal plugs were pregnant. The negative control group and the stress group were treated with control IgG. The experimental method referred to the literature (Blois S M, et al. Nature Medicine, 2007, 13(12):1450-1457), and embryonic development was detected. All drugs were intraperitoneally administered at a concentration of 20 μs per mouse per day.

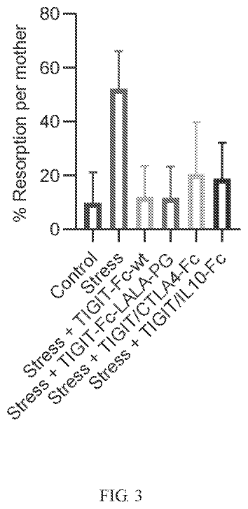

The experimental results are shown in FIG. 3. The abortion rate of each treatment group is significantly lower than that of the stress abortion group, indicating that the use of dimeric immunoadhesin has a good therapeutic effect.

### Example 5. The Effect of Dimeric Immunoadhesin on T Helper Cells

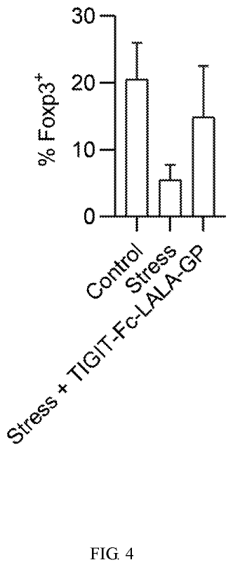

The para-aortic lymph nodes were separated from the mice in the control group, the stress group and the TIGIT-Fc-LALA-PG dimeric immunoadhesin treatment group, and the levels of Foxp3-positive T helper lymphocytes therein were detected. The separation and detection methods were the same as those in the literature (Kim B J, et al. Proceedings of the National Academy of Sciences, 2015, 112(5): 1559-1564). The results are shown in FIG. 4, and the results show the administration of TIGIT-Fc-LALA-PG dimeric immunoadhesin can effectively increase the level of Foxp3-positive T helper lymphocytes.

### Example 6. The Effect of Dimeric Immunoadhesin on Endometrial Receptivity after Endometrial Injury in Mice

An endometrial injury model was established in ICR mice by negative pressure uterine aspiration. The 8-week-old mice were divided into a uterine aspiration group, uterine aspiration+dimeric immunoadhesin treatment groups, and a blank control group. Each group contained ten mice. The modeling methods in the uterine aspiration group and the uterine aspiration+dimeric immunoadhesin treatment groups were the same as those in the literature (Wang Y P, et al. Journal of Zhejiang University: Medicine Edition, 2017(46): 191).

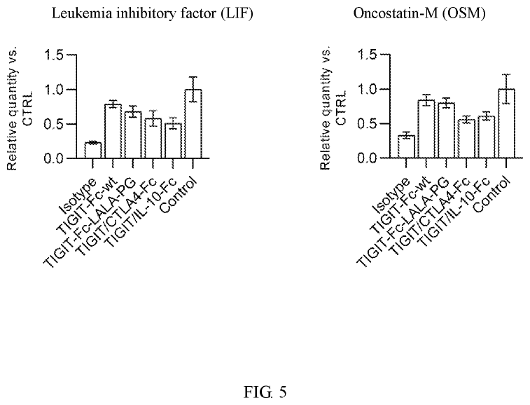

After the model was established, each administration group started to administer, and all drugs were intraperitoneally administered at a concentration of 20 μg per mouse per day. The uterine aspiration group was given a control antibody. Two weeks later, the drug was withdrawn for one week, and then the estrus was determined according to the vaginal smear. The male and female mice were caged at 1:1 that night, and the vaginal plug was checked at 7:00 a.m. the next morning. Those with vaginal plug were recorded as pregnant for 0.5 days. Each group was tested for endometrial receptivity. The test method was the same as that in the literature. The levels of leukemia inhibitory factor (LIF) and oncostatin (OSM) in the tissues were tested by enzyme-linked immunosorbent assay (ELISA). The window period of the endometrial receptivity in mice was about 4 days after conception. The expression of LIF and OSM, which are endometrial receptivity markers of the pregnant mice, is shown in FIG. 5. The results show that dimeric immunoadhesin therapy can effectively alleviate the endometrial injury caused by uterine aspiration.

### Example 7. The Effect of Dimeric Immunoadhesin on Intrauterine Adhesions in Mice

The 8-week-old ICR mice were divided into an intrauterine adhesions group, an intrauterine adhesion+dimeric immunoadhesin treatment group, and a blank control group. Each group contained 10 mice. The intrauterine adhesions group and the intrauterine adhesions+dimeric immunoadhesin treatment group were subjected to intrauterine adhesion modelling. The modeling method was as follows: the night before the operation, the mice were deprived of food but not water for 12 h; after anesthesia, the lower abdomen was routinely sterilized, and a midline incision was made to expose the Y-shaped uterus; using a 1 mL syringe, a needle was inserted into the uterine cavity at the uterine pelvis, and facing both sides 50 μL of 25% phenol mucilage was slowly injected in the direction of each ovary.

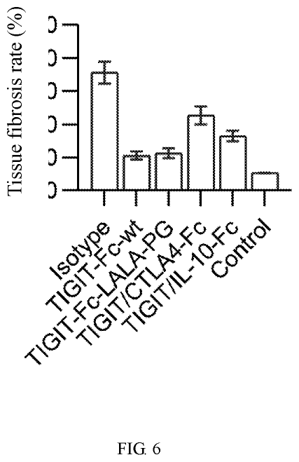

After the modeling was completed, the abdomen was closed in layers and the field of operation was disinfected. After the model was established, the control group was injected with normal saline, each administration group started to administer, and the intrauterine adhesions group was given control antibody. All drugs were intraperitoneally administered at a concentration of 20 μg per mouse per day. The mice were sacrificed to evaluate the degree of uterine fibrosis in the mice 18 days after continuous administration. According to the results in FIG. 6, the dimeric immunoadhesin therapy can effectively relieve the formation of endometrial and subendometrial fibrotic tissue.

In summary, in the mouse model of spontaneous abortion, dimeric immunoadhesin has excellent therapeutic effects on maternal-fetal immune tolerance disorders and diseases related to decreased endometrial receptivity, which is conducive to the conduct of subsequent clinical trials.

What is claimed is:

1. A soluble dimeric immunoadhesin, comprising a dimerized first polypeptide chain and a dimerized second polypeptide chain, wherein the first polypeptide chain has a general formula of Z1-Z2, and the second polypeptide chain has a general formula of Y1-Y2,
wherein Z1 is (i) an extracellular domain of a first cell surface receptor or a functional variant or fragment thereof, or (ii) a first cytokine or a functional variant or fragment thereof; Z2 is a dimerization domain or a functional variant or fragment thereof; Y1 is (i) an extracellular domain of a second cell surface receptor or a functional variant or fragment thereof, or (ii) a second cytokine or a functional variant or fragment thereof; and Y2 is a dimerization domain or a functional variant or fragment thereof.

2. The soluble dimeric immunoadhesin according to claim 1, wherein,
the Z1 and the Y1 are the same or different extracellular domains or functional variants or fragments thereof, and each being any one selected from the group consisting of: 4-1BB; ACTH receptor; activin receptor; BLTR (leukotriene B4 receptor); BMP receptor; C3a receptor; C5a receptor; CCR1; CCR2; CCR3; CCR4; CCR5; CCR6; CCR7; CCR8; CCR9; CD19; CD22; CD27; CD28; CD30; CD40; CD70; CD80; CD86; CD96; CD200R; CTLA-4; CD226; CD274; CD273; CD275; CD276; CD278; CD279; VSTM3 (TIGIT, B7R1); CD112; CD155; B7H6; NKp30; ICAM; VLA-4; VCAM; CT-1 receptor; CX3CR1; CXCR1; CXCR2; CXCR3; CXCR4; CXCR5; D6; DARC; DcR3; DR4; DR5; DcR1; DcR2; ECRF3; Fas; fMLP receptor; G-CSF receptor; GIT receptor; GM-CSF receptor; growth hormone receptor; HVEM; BTLA; interferon-α receptor; interferon-β receptor; interferon-γ receptor; IL-1 receptor type I; IL-1 receptor type II; IL-10 receptor; IL-11 receptor; IL-12 receptor; IL-13 receptor; IL-15 receptor; IL-16 receptor (CD4); IL-17 receptor A; IL-17 receptor B; IL-17 receptor C; IL-17 receptor D; IL-17 receptor E; IL-18 receptor; IL-2 receptor; IL-3 receptor; IL-4 receptor; IL-5 receptor; IL-6 receptor; IL-7 receptor; IL-9 receptor; IL-20 receptor A; IL-20 receptor B; IL-21 receptor; IL-22 receptor A; IL-22 receptor B; IL-28 receptor A; IL-27 receptor A; IL-31 receptor A; BCMA; TACI; BAFF receptor; immunomodulatory semaphoring receptor CD72; Kaposi's sarcoma-associated herpesvirus GPCR; lipoxin A4 receptor; lymphotoxin β receptor; lysophospholipid growth factor receptor; neurokinin 1; μ-, δ-, and κ-opioid receptors of endorphins; oncostatin M receptor; osteopontin receptor; osteoprotegerin; Ox40; OX40L; PACAP and VIP receptors; PAF receptor; poxvirus; IFNα/β receptor homologs; poxvirus IFNγ receptor homologs; poxvirus IL-10 receptor homologs; poxvirus membrane-bound G protein-coupled receptor homologs; poxvirus-secreted chemokine binding protein; poxvirus TNF receptor homologs; prolactin receptor; RANK; RON receptor; SCF receptor; somatostatin receptor; T1/ST2; TGF-β receptor; TNF receptor; TNFRSF19; TPO receptor; US28; XCR1; erythropoietin receptor; growth hormone receptor; leukemia inhibitory factor receptor; and C-kit receptor.

3. The soluble dimeric immunoadhesin according to claim 1, wherein.
the Z1 and the Y1 are the same or different cytokines or functional variants or fragments thereof, and each being any one selected from the group consisting of: α-MSH; 9E3/cCAF; ACTH; activin; AK155; angiogenesis inhibitor; Apo2L/TRAIL; APRIL; BAFF; BLR1 ligand/BCA-1/BLC/CXCL13; BMP family; BRAK; calcitonin gene-related peptide; molluscum contagiosum virus CC chemokine; CCL27; CCL28; CD100/Sema4D; CD27 ligand; CD30 ligand; CD40 ligand; CKβ8-1/MPIF-1/CCL23; CLF/CLC; CSF-1; CT-1; CTAP-III, βTG and NAP-2//CXCL7; CXCL16; defensins; ELC/MIP-3β/Exodus-3/CCL19; ENA-78/CXCL5; endorphins; endostatin; eosinophil chemotactic factor 2/MPIF-2/CCL24; eosinophil chemotactic factor/CCL11; erythropoietin; Exodus-1/LARC/MIP-3α; Fas ligand; Flt-3 ligand; fMLP; Fractalkine/CX3CL1; G-CSF; GCP-2/CXCL6; GM-CSF; growth hormone; HCC-1/CCL14; HCC-4/CCL16; high-mobility group box 1; human cathelicidin antimicrobial peptide LL-37; I-309/CCL1; IFNα, IFNβ and IFNω ligands; IFNγ; IL-1α; IL-1β; IL-10; IL-11; IL-12; IL-13; IL-15; IL-16; IL-17A; IL-17B; IL-17C; IL-17D; IL-17E; IL-17F; IL-18; IL-1Ra; IL-2; IL-27; IL-3; IL-4; IL-5; IL-6; IL-7; IL-8/CXCL8; IL-9; IP-10/CXCL10; IL-19; IL-20; IL-21; IL-22; IL-23; IL-24; IL-26; IL-31; keratinocyte growth factor; KSHV-associated IL-6 ligand; leptin; leukotaxin 1/HCC-2/MIP-1δ/CCL15; leukotriene B4; LIGHT; lipoxin; chemotactic factor for lymphocyte (ChFL)/XCL1; lymphotoxins a and (3; lysophospholipid growth factor; macrophage-derived chemokine; macrophase-stimulating protein; MCP-1/CCL2, MCP-2/CCL8, MCP-3/CCL7, MCP-4/CCL13, and MCP-5/CCL12; methoxyestradiol; MGSA/GRO/CXCL1, CXCL2, and CXCL3; MIF; MIG/CXCL9; MIP-1a/CCL3 and MIP-1β/CCL4; MIP-1γ/MRP-2/CCF18/CCL9/10; MuC10/CCL6; oncostatin M; osteopontin; parapoxvirus IL-10 homologs; PARC/DC-CCK1/AMAC-1/CCL18; PDGF-A; PDGF-B; PDGF-C; PDGF-D; platelet activating factor; platelet factor 4/CXCL4; poxvirus growth factor related to epidermal growth factor; poxvirus-secreted complement regulatory protein; poxvirus vascular endothelial growth factor homologs of orf virus; prolactin; RANK ligand; RANTES/CCL5; S100A12; SDF-1/CXCL12; SERP-1, secreted poxvirus serine protease inhibitor; SLC/Exodus-2/TCA-4/CCL21; somatostatin; stem cell factor; substance P; TARC/CCL17; TCA3/mouse CCL1; TECK/CCL25; TGFβ; thrombopoietin; TNFα; TSG-6; TWEAK; vaccinia virus semaphorin; vCXC-1 and vCXC-2; VEGF; VIP and PACAP; and viral IL-10 variants.

4. The soluble dimeric immunoadhesin according to claim 1, wherein,
the Z2 and the Y2 are Fc fragments of IgG or Fc mutants that change biological activity thereof, or heterodimeric IgG-Fc fragments constructed using Knob-in-holes technology, ART-Ig technology that changes charge polarity, or BiMab technology, and flexible linker can be added if necessary.

5. The soluble dimeric immunoadhesin according to claim 1, wherein,
when each of the Z1 and the Y1 is an extracellular domain of TIGIT or a functional variant or fragment thereof, amino acid sequences of the Z1 and the Y1 are at least 90% identical to an amino acid sequence shown in SEQ ID NO: 1; and
the soluble dimeric immunoadhesin has an amino acid sequence shown in SEQ ID NO: 2 or SEQ ID NO: 3.

6. The soluble dimeric immunoadhesin according to claim 1, wherein,
the Z1 is an extracellular domain of TIGIT or a functional variant or fragment thereof, the Y1 is an extracellular domain of CTLA4 or a functional variant or fragment thereof, an amino acid sequence of the Z1 is at least 90% identical to the amino acid sequence shown in SEQ ID NO: 1, and an amino acid sequence of the Y1 is at least 90% identical to an amino acid sequence shown in SEQ ID NO: 4; and
the Z1-Z2 polypeptide chain comprises an amino acid sequence shown in SEQ ID NO: 5, and the Y1-Y2 polypeptide chain comprises an amino acid sequence shown in SEQ ID NO: 6.

7. The soluble dimeric immunoadhesin according to claim 1, wherein,
the Z1 is an extracellular domain of TIGIT or a functional variant or fragment thereof, the Y1 is cytokine IL-10 or a functional variant or fragment thereof, the amino acid sequence of the Z1 is at least 90% identical to the amino acid sequence shown in SEQ ID NO: 1, the amino acid sequence of the Y1 is at least 90% identical to an amino acid sequence shown in SEQ ID NO: 7; and
the Z1-Z2 polypeptide chain comprises the amino acid sequence shown in SEQ ID NO: 5, and the Y1-Y2 polypeptide chain comprises an amino acid sequence shown in SEQ ID NO: 8.

8. A pharmaceutical composition comprising the soluble dimeric immunoadhesin according to claim 1, further comprising a medically acceptable pharmaceutical carrier.

9. Use of the soluble dimeric immunoadhesin according to claim 1 in the preparation of a medicine for the treatment and prevention of infertility-related diseases.

10. The use of the soluble dimeric immunoadhesin in the preparation of a medicine for the treatment and prevention of infertility-related diseases according to claim 9, wherein,
the infertility-related diseases comprise diseases related to maternal-fetal immune tolerance disorder or gynecological reproductive inflammation.

11. The use of the soluble dimeric immunoadhesin in the preparation of a medicine for the treatment and prevention of infertility-related diseases according to claim 10, wherein,
the diseases related to maternal-fetal immune tolerance disorder comprise recurrent spontaneous abortion, threatened abortion, or treatment failure of assisted reproductive technology; and
the diseases related to gynecological reproductive inflammation comprise pelvic inflammatory disease, decreased endometrial receptivity, endometritis, endometrial polyps, intrauterine adhesions, reduction of endometrial glands, endometrial fibrosis, amenorrhea, abnormal uterine bleeding, adenomyosis and endometriosis, reproductive system infection or hysteromyoma.


SEQUENCE LISTING (13 sequences)

SEQ ID NO: 1 - protein, 120 aa, Artificial sequence
  Designed amino acid sequence of TIGIT protein
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 2 - protein, 352 aa, Artificial sequence
  Designed amino acid sequence of TIGIT immunoadhesin
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 352 aa, Artificial sequence
  Designed amino acid sequence of TIGIT immunoadhesin
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 125 aa, Artificial sequence
  Designed amino acid sequence of ABATACEPT N terminal domain
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQ

SEQ ID NO: 5 - protein, 352 aa, Artificial sequence
  Designed amino acid sequence of dimer immunoadhesin
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 6 - protein, 357 aa, Artificial sequence
  Designed amino acid sequence of dimer immunoadhesin
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 160 aa, Artificial sequence
  Designed amino acid sequence acting as cell factor
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 8 - protein, 392 aa, Artificial sequence
  Designed amino acid sequence acting as cell factor
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 1056 bp, Artificial sequence
  Designed nucleotide sequence of dimer immunoadhesin
atgatgaccggcaccatcgagaccaccggcaacatcagcgccgagaagggcggcagcatcatcctgcagtgccacctgagcagcaccaccgcccaggtgacccaggtgaactgggagcagcaggaccagctgctggccatctgcaacgccgacctgggctggcacatcagccccagcttcaaggacagagtggcccccggccccggcctgggcctgaccctgcagagcctgaccgtgaacgacaccggcgagtacttctgcatctaccacacctaccccgacggcacctacaccggcagaatcttcctggaggtgctggagagcagcgtggccgagcacggcgccagattccagatccccgagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 10 - DNA, 1056 bp, Artificial sequence
  Designed nucleotide sequence of dimer immunoadhesin
atgatgaccggcaccatcgagaccaccggcaacatcagcgccgagaagggcggcagcatcatcctgcagtgccacctgagcagcaccaccgcccaggtgacccaggtgaactgggagcagcaggaccagctgctggccatctgcaacgccgacctgggctggcacatcagccccagcttcaaggacagagtggcccccggccccggcctgggcctgaccctgcagagcctgaccgtgaacgacaccggcgagtacttctgcatctaccacacctaccccgacggcacctacaccggcagaatcttcctggaggtgctggagagcagcgtggccgagcacggcgccagattccagatccccgagcccaagagctgcgacaagacccacacctgccccccctgccccgcccccgaggccgccggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgggcgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 11 - DNA, 1056 bp, Artificial sequence
  Designed nucleotide sequence of dimer immunoadhesin
atgatgaccggcaccatcgagaccaccggcaacatcagcgccgagaagggcggcagcatcatcctgcagtgccacctgagcagcaccaccgcccaggtgacccaggtgaactgggagcagcaggaccagctgctggccatctgcaacgccgacctgggctggcacatcagccccagcttcaaggacagagtggcccccggccccggcctgggcctgaccctgcagagcctgaccgtgaacgacaccggcgagtacttctgcatctaccacacctaccccgacggcacctacaccggcagaatcttcctggaggtgctggagagcagcgtggccgagcacggcgccagattccagatccccgagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtgcaccctgccccccagcagagacgagctgaccaagaaccaggtgagcctgagctgcgccgtgaagggcttctaccccagcgacatcgccgtggagtgggagagcaacggccagcccgagaacaactacaagaccaccccccccgtgctggacagcgacggcagcttcttcctggtgagcaagctgaccgtggacaagagcagatggcagcagggcaacgtgttcagctgcagcgtgatgcacgaggccctgcacaaccactacacccagaagagcctgagcctgagccccggcaag

SEQ ID NO: 12 - DNA, 1071 bp, Artificial sequence
  Designed nucleotide sequence of dimer immunoadhesin
atgcacgtggcccagcccgccgtggtgctggccagcagcagaggcatcgccagcttcgtgtgcgagtacgccagccccggcaaggccaccgaggtgagagtgaccgtgctgagacaggccgacagccaggtgaccgaggtgtgcgccgccacctacatgatgggcaacgagctgaccttcctggacgacagcatctgcaccggcaccagcagcggcaaccaggtgaacctgaccatccagggcctgagagccatggacaccggcctgtacatctgcaaggtggagctgatgtacccccccccctactacctgggcatcggcaacggcacccagatctacgtgatcgaccccgagccctgccccgacagcgaccaggagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgcccccctgcagagacgagctgaccaagaaccaggtgagcctgtggtgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 13 - DNA, 1176 bp, Artificial sequence
  Designed nucleotide sequence of dimer immunoadhesin
agccccggccagggcacccagagcgagaacagctgcacccacttccccggcaacctgcccaacatgctgagagacctgagagacgccttcagcagagtgaagaccttcttccagatgaaggaccagctggacaacctgctgctgaaggagagcctgctggaggacttcaagggctacctgggctgccaggccctgagcgagatgatccagttctacctggaggaggtgatgccccaggccgagaaccaggaccccgacatcaaggcccacgtgaacagcctgggcgagaacctgaagaccctgagactgagactgagaagatgccacagattcctgccctgcgagaacaagagcaaggccgtggagcaggtgaagaacgccttcaacaagctgcaggagaagggcatctacaaggccatgagcgagttcgacatcttcatcaactacatcgaggcctacatgaccatgaagatcagaaacgagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgcccccctgcagagacgagctgaccaagaaccaggtgagcctgtggtgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag